(19) United States
(12) Patent Application Publication
(10) Pub. No.: US 2026/0115476 A1
(43) Pub. Date: Apr. 30, 2026

(54) Methods And Systems For Automated Detection Of Neuropsychiatric Disorders

(71) Applicant: The Trustees of the University of Pennsylvania, Philadelphia, PA (US)

(72) Inventors: Juan Francisco Lopez Luna, Philadelphia, PA (US); Flavia Vitale, Wilmington, DE (US); Bijan Pesaran, Villanova, PA (US); Andrew G. Richardson, Wynnewood, PA (US)

(21) Appl. No.: 19/368,370

(22) Filed: Oct. 24, 2025

Related U.S. Application Data
(60) Provisional application No. 63/711,499, filed on Oct. 24, 2024.

(51) Int. Cl. A61N 1/36

(52) U.S. Cl. CPC A61N 1/36139; A61N 1/36082

(57) ABSTRACT

Methods and systems for automated detection of neuropsychiatric disorders are described herein. In one aspect, a method can include measuring, via a wearable device of a diagnostic system, one or more electrical signals corresponding to muscle movement of a user, wherein the wearable device comprises one or more MXene-comprising sensors; inputting information indicative of the one or more electrical signals into a trained model; determining, by the trained model and based on the inputted information, whether the user is experiencing symptoms of a neuropsychiatric disorder; and sending information indicative of the determining to a component of the diagnostic system.

### CROSS-REFERENCE TO RELATED APPLICATIONS

This application claims the benefit of and priority to U.S. Provisional Patent Application No. 63/711,499 titled “Methods and Systems for Automated Detection of Neuropsychiatric Disorders” filed Oct. 24, 2024, the entirety of which is incorporated herein by reference for any and all purposes.

### TECHNICAL FIELD

The present disclosure relates to the field of surface electromyography and to the field of neuropsychiatric disorder detection.

### BACKGROUND

Behavioral evaluation of clinical states for neuropsychiatric disorders is primarily performed by clinicians or in the form of self-reports using questionnaires, like the Yale-Brown Obsessive-Compulsive Scale (YBOCS) for Obsessive Compulsive Disorder (OCD) or the Yale Global Tic Severity Scale (YGTSS) for tic severity assessment in Tourette's Syndrome (TS). However, these metrics are subjective, taken at discrete intervals (once a week or once per month), and most of the time, require an expert to validate them.

Wearable technology improvements have enabled continuous and real-time physiological and behavioral data monitoring in patients with neuropsychiatric disorders.

Studies have shown that it is feasible to use wearables in naturalistic environments during relevant behavior in individuals with obsessive-compulsive disorder (OCD). Additionally, researchers have utilized the Apple Watch to record tremors and uncontrolled movements in people with Parkinson's Disease.

Surface Electromyography (sEMG) is a technique that uses electrodes placed on the skin's surface to record neuromotor activity during muscle activation. sEMG has been used in neuroprosthetic research to decode motor intentions for prosthetic arm control and, more recently, in the consumer space by META as a brain-computer interface to infer hand gestures to control cursors and handwriting decoding. In the neuropsychiatric space, there have been some studies using sEMG to predict treatment outcome in depression through facial expressions; for eating disorders, one study recorded sEMG from eating muscles to provide real-time feedback to promote mindful eating; another study used EMG to detect tics in Tourette Syndrome, and automated, real-time detection of tonic-seizures using a wearable EMG for epilepsy. The important role in these applications of real-time feedback and integration with other clinically-relevant data streams such as neural recordings and stimulation control using implanted electrodes, means that the wearable sensor data stream also needs to be integrated with a software tool to permit distributed synchronization and interpretation/decoding.

However, translating sEMG wearable devices into the clinical space is challenging since high-quality signals require electrodes to have high stretchability to be conformational to the skin surface and high conductivity. Current commercial electrodes, such as Ag/AgCl, are not suitable for long-term recordings since they need a gel interface that is bulky, uncomfortable, and can cause a skin rash. Dry electrodes, and more recently, metal stretchable electrodes, have overcome these issues, but they are not scalable, and there hasn't been wide adoption yet.

### SUMMARY

Methods and systems for automated detection of neuropsychiatric disorders are described herein. In one aspect, a method can include measuring, via a wearable device of a diagnostic system, one or more electrical signals corresponding to muscle movement of a user, wherein the wearable device comprises one or more MXene-comprising sensors; inputting information indicative of the one or more electrical signals into a trained model; determining, by the trained model and based on the inputted information, whether the user is experiencing symptoms of a neuropsychiatric disorder; and sending information indicative of the determining to a component of the diagnostic system.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIG. 1. A) MXene-based electrode array connected to the wireless acquisition device. B) Top, sleeve placed on the forearm using anatomical landmarks. Bottom, PPG, EMG (2 channels shown), and IMU signals recorded wirelessly with the device. C) Symptom provocation experiment design. D) Patient-reported SUDS (mean +/− sd) measured during symptom provocation. Left: Effective compulsion reduces SUDS to 10 or below following provocation. Right: Ineffective compulsion does not reduce SUDS to 10 or below following provocation. E) t-SNE dimensionality reduction of SmartSleeve EMG data for individual 40 s feature integration windows recorded during compulsion and ADL trials. Left: Effective compulsion (orange) can be accurately discriminated from ADL (blue). Right: Ineffective compulsion (yellow) cannot be accurately discriminated from ADL (blue).

FIG. 2. A) Top, back of the sleeve prototype with dry MXene electrodes. Bottom, sleeve placed on the forearm using anatomical landmarks. B) Top, photographs of the front and back of the device with labeled components. Bottom, PPG, EMG (2 channels), and IMU signals recorded wirelessly with the device. C) Symptom provocation experiment design. D) Patient-reported SUDS (mean +/− sd) measured during symptom provocation. Left: Effective compulsion reduces SUDS to 10 or below following provocation. Right: Ineffective compulsion does not reduce SUDS to 10 or below following provocation. E) t-SNE dimensionality reduction of SmartSleeve EMG data for individual 40 s feature integration windows recorded during compulsion and ADL trials. Left: Effective compulsion (orange) can be accurately discriminated from ADL (blue). Right: Ineffective compulsion (yellow) cannot be accurately discriminated from ADL (blue).

FIG. 3. Technology overview. A) Current approaches. B) Proposed smart wearable platform featuring a coil and sleeve. C) Multimedia sensor array for coil and sleeve. D) Computational framework connecting systems (EMAs) to physiology (EMG, PPG, EDA, IMU). Models can be fit to predict patient's activity profile with training data from symptom provocations. Brain recordings can be triggered when SmartSleeve activity profile enters states labelled high compulsivity or activities of daily living. Models can be updated with rating and EMAs provided by patients.

FIG. 4. A) Nucleus Accumbens (NAc) and ventral putamen (VeP) targets. B) Changes in SUDS over time.

FIG. 5. Dry eEMG MXene technology. Custom designs for A) small 5 mm and B) medium 8 mm electrode size.

FIG. 6. Sample EMG validation traces. A) Acceptable fitting. SNR=18 dB. B) Poor fitting. SNR=8.3 dB.

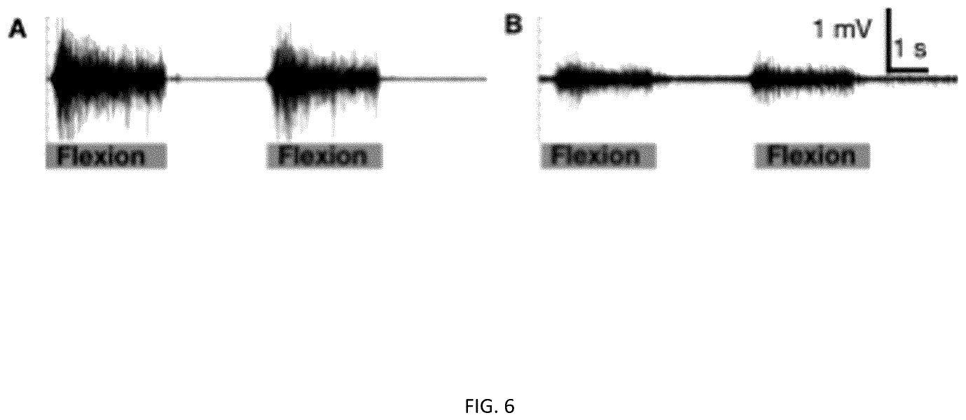

FIG. 7: Examples of MXene electrodes.

FIG. 8: Graph of impedance vs. frequency for different electrode examples.

FIG. 9: Graph of mean signal-to-noise ratio (SNR) for corresponding channels of MXene-based electrodes.

FIG. 10: Example wireless acquisition device that collects and transmits sEMG and inertial data.

FIG. 11: Data processing system using a nested sliding window to classify effective compulsions.

FIG. 12: Electrode placement based on forearm zones.

FIG. 13: Zone mapping and corresponding electrode array design.

FIG. 14: Discrete hand gesture acquisition protocol.

FIG. 15: Recording process for electrodes.

FIG. 16: SNR measurement for electrode system.

FIG. 17: Data analysis pipeline.

FIG. 18: High-pass filtered EMG signals.

FIG. 19: Median SNR per muscle gestures used per channel.

FIG. 20: Median SNR per channel, and corresponding predicted medians.

FIG. 21: SNR change across a number of days.

FIG. 22: Time-domain features projections.

FIG. 23: Classification performance over time.

FIG. 24: Baseline models for electrode shift compensation. The process included processing raw sEMG data (50-450 Hz) band-pass Butterworth filter (4^{th} order), storing the processed data locally to be loaded by a trainer. For each training session: data splitting: take one position (randomly selecting one trial for validation) and one position for testing-pairwise; data segmentation: segment each trial in windows of 250 ms with 225 ms overlap; normalize the data by channel using a training scaler; and wrap in pytorch data loaders. Trained two models: logistic regression (repeat each training 10 times) and 1D CNN (set to 5 epochs and run 5 times).

FIG. 25: Graphs of electrode shift experiments: Multi-position electrode placement within the same skin zone. 4 trials for each position were taken for training (1 for validation) and 1 trial from other positions for test (pairwise). The data was randomized and repeated 10 times.

FIG. 26: Graph corresponding to electrode shift experiments. The results suggest that the model can accurately predict if the sleeve is placed within the skin zone range.

FIG. 27: Calibration process.

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” can include the embodiments “consisting of” and “consisting essentially of.” The terms “comprise(s),” “include(s),” “having,” “has,”“can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant FIGs. and numerical values which differ from the stated value by less than the experimental error of conventional measurement technique of the type described in the present application to determine the value.

All ranges disclosed herein are inclusive of the recited endpoint and independently of the endpoints. The endpoints of the ranges and any values disclosed herein are not limited to the precise range or value; they are sufficiently imprecise to include values approximating these ranges and/or values.

As used herein, approximating language can be applied to modify any quantitative representation that can vary without resulting in a change in the basic function to which it is related. Accordingly, a value modified by a term or terms, such as “about” and “substantially,” may not be limited to the precise value specified, in some cases. In at least some instances, the approximating language can correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” can refer to plus or minus 10% of the indicated number. For example, “about 10%” can indicate a range of 9% to 11%, and “about 1” can mean from 0.9-1.1. Other meanings of “about” can be apparent from the context, such as rounding off, so, for example “about 1” can also mean from 0.5 to 1.4.

Further, the term “comprising” should be understood as having its open-ended meaning of “including,” but the term also includes the closed meaning of the term “consisting.” For example, a composition that comprises components A and B can be a composition that includes A, B, and other components, but can also be a composition made of A and B only. Any documents cited herein are incorporated by reference in their entireties for any and all purposes.

Any embodiment or aspect provided herein is illustrative only and does not limit the scope of the present disclosure or the appended claims. Any part or parts of any one or more embodiments or aspects can be combined with any part or parts of any one or more other embodiments or aspects.

Methods and systems for automated neuropsychiatric symptom detection are described herein. In one example, a novel wearable device is disclosed, using fabric electrodes inked with a highly conductive nanomaterial to be placed in the forearm and acquire neuromotor signals across the forearm circumference. Further, a time-series machine learning algorithm is provided that allows for discrimination of compulsions from activities of daily living (ADL) in data from a pilot experiment from a patient with OCD. Together, they form a wearable neuropsychiatric platform called Smart Sleeve. The SmartSleeve allows for sEMG signals from the forearm or other body part to be collected and monitored for symptomatic states in people with OCD.

Information from Smart Sleeve can also be used to inform and control brain stimulation therapy, by synchronizing with and triggering an implantable device upon detection of symptom onset synchronized in real-time. Thus, the platform can include 1) a wearable device (Smart Sleeve), 2) the algorithm to discriminate symptoms from ADLs and other movements, 3) a real-time software tool for synchronization and control of the functions of a 4) an adaptive implanted neurostimulation device.

A wearable, adaptive recording-and-stimulation Smart Sleeve platform has potential applications for depression, Tourette Syndrome, Parkinson's, and movement disorders and is ideally suited for obsessive-compulsive disease (OCD). OCD symptoms feature overt, recognizable movements, most relevant to compulsive dimensions of the disease, as well as covert, less-recognizable affective states, most relevant to obsessive dimensions of the disease.

Compulsive behaviors can be severe, lasting three or more hours per day, during which time patients feel driven to perform extended rituals of functional and non-functional acts according to specific rules or in a stereotyped fashion. For example, when a patient with a checking compulsion is asked to unlock a door to enter a room, the functional acts to insert the key, turn the lock, and open the door are embedded within non-functional acts to visit objects and locations in a stereotyped ritual repeatedly. An isolated act lasting seconds becomes a ritual lasting more than a minute.

The current state of the art in wearables for OCD symptom monitoring uses wrist-worn commercially-available devices with (1) inertial measurement units (IMUs) that, through machine learning methods, attempt to infer compulsive actions and (2) photoplethysmography (PPG) sensors that measure heart rate variability (HRV). Features extracted from HRV negatively correlate with stress.

These wearables have been tested in small pilot studies with OCD patients or control subjects engaged in scripted compulsive actions or stress-inducing tasks. Testing has been confined to a laboratory setting in these studies. The wearables remain far from clinical adoption. The sensors in standard commercial products (e.g., smart watches) are not designed to detect compulsions, which is a limiting factor.

The systems and methods described herein provide low-cost and scalable wearable device that is capable of continuously acquiring high-quality EMG signals, and the described machine-learning model can discriminate between compulsions and activities of daily living. The Smart Sleeve platform is wearable technology that uses neuromotor activity to discriminate clinically relevant behavior or states in people with a neuropsychiatric disorder.

### Device

The device can include a flexible electrode array. The flexible electrode array can be fabricated based on technology for high-resolution and sensitivity dry surface EMG. The sEMG electrodes, composed of Ti3C2Tx MXene, a carbon-based 2D material, offer enhanced conductivity, electrochemical impedance, and mechanical compliance compared to current technology. This makes it ideally suited to sensing body bioelectrical activity. Moreover, Ti3C2Tx MXene is cheaper to source and process and has 4-10× lower impedance, which translates into higher sensitivity and better signal-to-noise characteristics without the need for any gel or harsh skin treatment. Importantly, these arrays can be used for continuous monitoring, as the impedance with the skin may not vary during continuous wearing on the forearm for over two days, ensuring reliable data collection.

The system or platform can also include a custom-made battery-powered wireless acquisition device. The small (e.g., one-by-half-inch) device comprises commercial integrated circuits (IC) for signal acquisition and data transmission. It includes three main modules: the electromyography (EMG) acquisition module, the inertial measurement unit (IMU), and the control and Bluetooth transmitter module. The EMG data is acquired using an Intan RHD amplifier (RHD2216); the module is wired to a flexible flat cable (FFC) connector, enabling eight cEMG channel connections for single-ended or bipolar configuration recordings at a sampling frequency of 1 kHz. The IMU data is acquired using the low-power ICM-20948 chip from TDK, configured for ±500 dps on the gyroscope and +4 g on the accelerometer. The microcontroller and the Bluetooth transceiver module can be within the same integrated circuit. It contains a 128 MHz ARM cortex microcontroller that can communicate with the EMG and IMU modules through SPI and I2C, collect the data, and transmit it through Bluetooth low energy (BLE) protocol. The estimated data rate is less than 300 kb/s, below the maximum BLE 5.0 rate of 2 Mb/s. The expected power consumption of the device can less than 20 mW; using a 500 mAh Li-ion battery can provide approximately 80 hours of device uptime.

### Machine Learning Algorithm

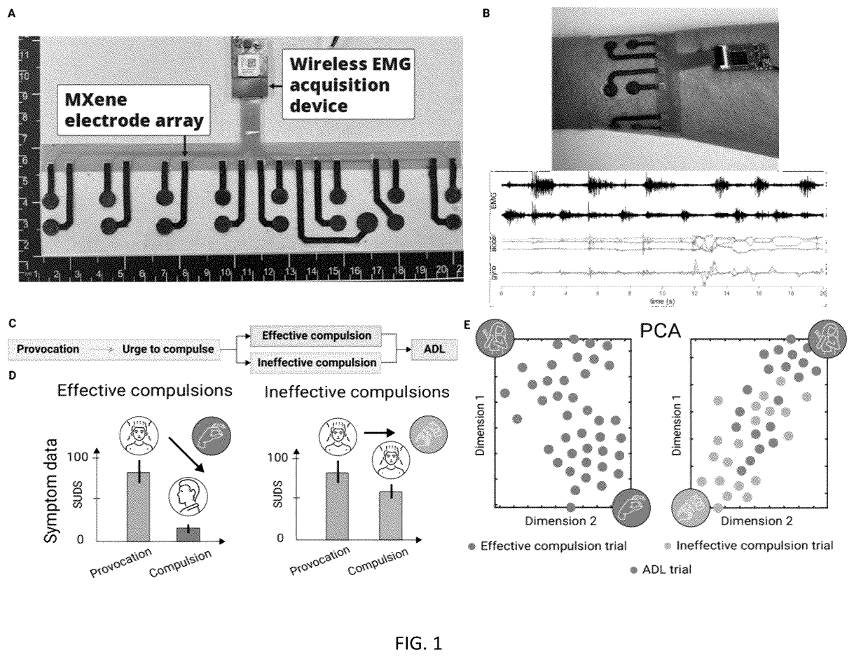

First, the EMG data can be analyzed to detect compulsions from ADL using standard approaches. The EMG activity can be band-pass filtered, 50-450 Hz, and rectified EMG activity. The root-mean-square power of EMG activity on each channel can be computed over a feature sample window of short duration. The EMG activity can then be concatenated from 50% overlapping feature sample windows in a longer feature integration window. We reduced the dimensionality of features in the feature integration window using PCA and selected the top set of modes as features for logistic regression. Activity from each feature integration window was classified as compulsion or ADL. Feature sample window duration, feature integration window, and PCA dimension were meta parameters selected by analyzing a validation data set. FIG. 1E parameters are a feature sample window equal to six seconds and a feature integration window of 40 seconds, using the first three dimensions from PCA. The classifier's accuracy can be estimated by the sensitivity and specificity average using cross-validation with a test set. Accuracy for effective compulsions can be 80%, and for ineffective compulsions can be 52%. FIG. 1E left presents data projected using t-SNE, a nonlinear data visualization method. A point for each feature integration window on trials with effective compulsions is shown in (red) or ADL (blue). FIG. 1E right presents the same for trials with ineffective compulsions (orange) or ADL (blue). The results suggest that the experimental design can reveal the potential power of the SmartSleeve technology to detect effective compulsions with an accuracy exceeding 75%.

### Potential Applications

The SmartSleeve can be utilized to: Provide real-time feedback for adaptive Deep Brain Stimulation (DBS) during relevant behaviors in patients with OCD and other neuropsychiatric disorders with a symptomatologic motor component; provide real-time feedback to patients undergoing Cognitive Behavioral Therapy, both in sessions and while the patient is at home; be used as a research tool to automatically trigger brain recordings during relevant behavior in a natural setting, which is not possible so far. This synchronization will enable researchers to understand the root causes of neuropsychiatric disorders and potentially discover novel biomarkers; and generate objective personal records to monitor treatment progression.

Development of a Wearable to Detect Compulsions from Muscle Activity

Obsessive-Compulsive Disorder (OCD) affects 1.1-1.8% of the world's population and is ranked as one of the most disabling illnesses by the World Health Organization (WHO). Approximately 40% of OCD patients do not respond to standard treatment. Deep Brain Stimulation (DBS) has emerged as a potential therapeutic solution. Nevertheless, outcomes are limited to approximately 66% response rates and are primarily done in an open-loop fashion. Studies have reported that close-loop stimulation yields promising outcomes. However, the monitoring requirements significantly impact the performance and battery life of the implanted device. The disclosure provided herein provides for the development of a wearable device with novel dry bioelectrodes using a conductive nanomaterial (Ti₃C₂Tₓ MXene) to continuously acquire high-quality muscle signals at the forearm to detect the performance of compulsions using machine learning. This framework can be extended to other neuropsychiatric disorders and has the potential to be used for real-time clinical interventions and drive close-loop neurostimulation.

### Methods

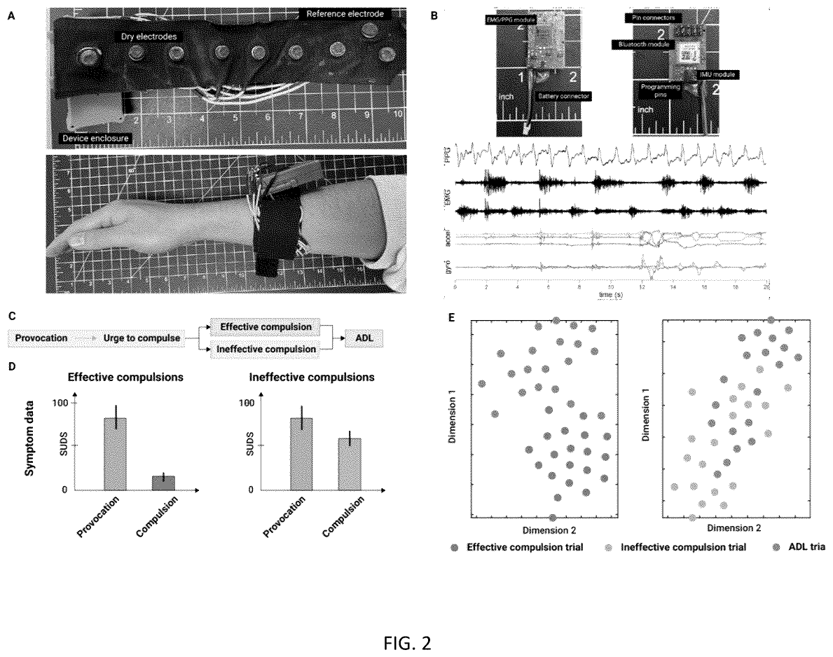

The wearable muscle sensing arrays can be fabricated from aqueous inks of Ti₃C₂Tₓ patterned on flexible laser-machined substrates (FIG. 2A). The electrodes can be “dry” (i.e., do not require any conductive gel to establish contact with the skin) and have properties comparable to commercial gel electrodes with high SNR for bioelectrical signal acquisition. A total of 16 electrodes (8 bipolar) can be arranged equidistant across the forearm. Using a custom wireless acquisition device, eight single-ended electromyography (EMG) channels and motion data can be captured with a 1 kHz sampling rate and transmit the data via Bluetooth (FIG. 2B). Data can be recorded from an OCD patient in a clinical setting, with the patient wearing the sleeve at the forearm during compulsion provocation experiment. To effectively discriminate compulsions from activities of daily living (ADL), a machine learning framework can be developed.

### Results and Conclusions

On each trial, the patient reported subjective units of distress (SUDS) and urge-to-compulse. Provocation trials elevated the patient's SUDS to 70, and after provocation, the patient reported an elevated urge-to-compulse of 80. Interestingly, symptom data revealed two groups of compulsive trials. Performing an effective compulsion would lower SUDS to ˜10 and reduce the urge-to-compulse through the ADL at the end of the trial. On the other hand, reported SUDS after the performance of an ineffective trial remained elevated at 50, and there was no reduction in the urge-to-compulse after doing the ADL to finish the trial.

The classifier performance can be estimated using average accuracy using cross-validation. For effective compulsion, the accuracy was 80%, and for ineffective compulsion, it was 53% (FIG. 2C). FIG. 2D shows the data projected into the PCA space for visualization, where clusters of effective compulsion can be distinguished from clusters of ADLs (left) but not with ineffective compulsions (right). The results suggest that the experimental design and the developed device can detect effective compulsions.

This work is a proof-of-concept for a wireless wearable device capable of detecting effective compulsions in patients with OCD, serving as a first-in-class objective symptom detection platform using EMG with the potential for closing the loop in DBS systems for the treatment of OCD and other psychiatric disorders.

### Development and Initial Testing of a Multimodal Wearable to Detect Compulsions From Electromyography (EMG)

OCD is a neuropsychiatric disorder characterized by the presence of persistent and intrusive thoughts (obsessions) and repetitive behaviors (compulsions), often performed to alleviate the anxiety generated by the obsessions. OCD affects 1.2% of U.S. adults, with reported impairment in half of the cases. Moreover, approximately 40% of OCD patients do not respond to standard treatment. Deep Brain Stimulation (DBS) has emerged as a potential therapeutic solution for OCD boosted by FDA approval via Humanitarian Device Exemption. Nevertheless, DBS is often conducted in an open-loop fashion, and outcomes are limited to approximately 66% response rates and are. Studies have reported that closed-loop stimulation yields promising outcomes for treating binge eating and OCD. However, the continuous monitoring requirements significantly impact the performance and battery life of the implanted DBS device. Here, we report a wearable sleeve device fabricated with Ti3C2Tx MXene to continuously acquire high-quality neuromotor signals at the forearm and detect the occurrence of effective compulsions in patients with mild to severe OCD using a machine learning approach.

### Material and Methods

We built a cotton lycra breathable sleeve with detachable snap buttons and custom-made dry electrodes fabricated with hydroxylated PVA aerogels coated with Ti3C2Tx MXene. The electrodes are arranged equidistant across the forearm. Using a wireless device, we captured eight single-ended MXene electromyography (EMG) channels at 1 kHz sampling rate. We transmitted the data via Bluetooth to a Python-based graphical user interface (GUI) laptop. We recorded single-patient data in a clinical setting, with the patient wearing the sleeve at the forearm and other clinical-grade BIOPAC sensors. The protocol consisted of repeating trials with equally spaced, two-minute blocks of symptom provocation, followed by the performance of effective or ineffective compulsions, and finally, an Activity of Daily Living (ADL) comparison movement. On each trial, the patient reported subjective units of distress score (SUDS) and urge-to-compulse, both in the 0-100 range. The start and stop times were flagged on each block using the BIOPAC software synchronized with the EMG data.

To accurately discriminate effective compulsions from ADL, we developed a machine learning framework. We preprocessed the data using a band-pass filter (50-450 Hz, 4th order Butterworth) and stored each block in a data structure. We used a long-duration feature integration window on each block for feature extraction. Within each of these windows, we computed the root-mean-square power of the EMG activity from each channel over a short-duration feature sample window with 50% overlap. After feature extraction, we reduced the dimensionality by applying PCA on the feature integration window and selected the top modes to feed a logistic regression model.

### Results/Discussions/Conclusions

On each trial, the patient reported SUDS and urge-to-compulse. Provocation trials elevated the patient's SUDS to 70, and after provocation, the patient reported an elevated urge-to-compulse of 80. Interestingly, symptom data revealed two groups of compulsive trials. Performing an effective compulsion would lower SUDS to ˜10 and reduce the urge-to-compulse through the ADL at the end of the trial. On the other hand, reported SUDS after the performance of an ineffective trial remained elevated at 50, and there was no reduction in the urge-to-compulse after doing the ADL to finish the trial.

Based on the machine learning model accuracy evaluation, we selected a feature sample window of 7 seconds and a feature integration window of 40 seconds. We estimate the classifier's performance by the average accuracy using cross-validation. For effective compulsion, the accuracy was 80%, and for ineffective compulsion, it was 53%. In FIG. 1E, we projected our data into the t-distributed Stochastic Neighbor Embedding (t-SNE) space for visualization, where we discerned clusters of effective compulsion from clusters of ADLs (left) but not with ineffective compulsions (right). Our results suggest that the experimental design and the developed device can detect effective compulsions.

Together, this work is a proof-of-concept for an MXene-based dry wearable device capable of detecting effective compulsions in patients with OCD, serving as a first-in-class objective symptom detection platform using EMG with the potential for closing the loop in DBS systems for the treatment of OCD and other psychiatric disorders.

### Manuscript

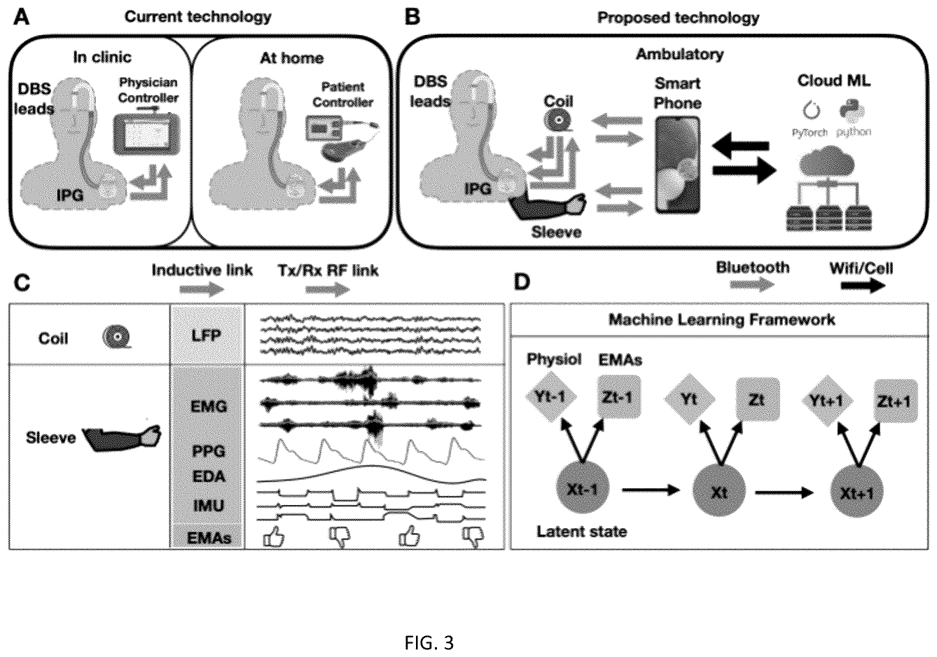

In deep brain stimulation (DBS), a neurostimulation device is implanted to affect circuits associated with neurological and neuropsychiatric disorders for potential therapeutic benefit. The U.S. Food and Drug Administration (FDA) has approved DBS for the treatment of refractory obsessive-compulsive disorder (OCD) through a Humanitarian Device Exemption (HDE) which brings relief to many but not all patients. Addressing the needs of the remaining cases requires new ways to introduce adaptive DBS (aDBS) paradigms, individualize biomarkers and refine brain perturbations. Our premise is that a smart wearable device with access to broad-band brain recordings in ambulatory patients is necessary to deliver effective next-generation DBS treatments for psychiatric illness. Our approach is illustrated in FIG. 3.

Today, clinical DBS systems typically permit neural activity streaming from the implanted electrodes under constrained settings. Streaming can be done in the clinic using a physician controller unit that also programs the implantable pulse generator (IPG; FIG. 3A). At home, patients use a patient controller that they hold near the IPG. This allows them to charge the IPG through an inductive link as well as to transfer neural recordings when clicking or swiping. This leads to several issues: 1) Recordings involve the patient handling the patient controller, a manual task which corrupts or alters the data. 2) The number and total duration of recordings is low because they require the patient to retrieve the patient controller and click/swipe. 3) Behavioral context is typically unavailable. 4) The equipment is bulky, too large and heavy to wear every day for truly ambulatory studies. 5) Raw neural signals are not usually streamed but compressed into biomarkers, spectral power sampled infrequently, every 1-10 minutes. These limits also apply to responsive approaches to DBS that do not use full-band neural signals and only control stimulation after compression. The temporal resolution of behavior is high-in the sub-seconds not minutes or tens of minutes. DBS electrodes provide high quality intracranial EEG (iEEG) recordings with ˜10 millisecond temporal precision. Harnessing brain-behavioral signals in an ambulatory setting offers the promise of many pay-offs including predicting symptoms, controlling stimulation and generating new treatment protocols⁵.

One strategy to brain-behavior synchronization is to perform behavioral and neural recordings continuously. However, overcoming the limits on neural recording with currently-implanted devices depends on substantial re-engineering of the implant. The problem is due to storage and energy. The battery powering the implantable pulse-generator (IPG) that performs the recordings has limited capacity and lifetime and so cannot continuously stream high-bandwidth data. Storing data on the IPG depends on a non-volatile form of magnetic memory, FRAM, which requires very little power but offers relatively little storage, typically <1 MB. However, constantly recording neural data may not be strictly necessary and analyzing large amounts of raw neural data can be expensive. Instead, we propose an innovative wearable approach (FIG. 3B). We propose to miniaturize the patient controller into a wearable coil that sits in a piece of clothing. The clothing will place the coil near the IPG in the chest. A small, lightweight coil dongle will communicate with a mobile device to receive commands and transmit raw neural data over a bluetooth RF link. We will pair the coil with a wearable sleeve that monitors patient-relevant behavioral and physiological signals to generate a symptom activity profile. A key to overcoming storage and energy limits without re-engineering the IPG is to engineer algorithms that select neural recording epochs based on a wearable symptom activity profile in a flexible, software-triggered manner—to make the sleeve smart.

A wearable, adaptive recording-and-stimulation SmartSleeve platform has potential applications to depression, Tourette Syndrome, Parkinson's and movement disorders, and is ideally suited for obsessive-compulsive disease (OCD). OCD symptoms feature overt, recognizable movements, most relevant to compulsive dimensions of the disease, as well as covert, less-recognizable affective states, most relevant to obsessive dimensions of the disease. Compulsive behaviors can be severe, lasting three or more hours per day, during which time patients feel driven to perform extended rituals of functional and non-functional acts according to certain rules or in a stereotyped fashion. For example, when a patient with a checking compulsion is asked to unlock a door to enter a room, the functional acts to insert the key, turn the lock and open the door are embedded within non-functional acts to repeatedly visit objects and locations in a stereotyped ritual. An isolated act lasting seconds becomes a ritual that can last more than a minute. This means patient-specific compulsive episodes can be readily discriminated from activities of daily living (ADL). FIG. 3C presents our strategy which features a multimodal sensor array and patient labels that we hypothesize will improve OCD symptom detection. We propose to focus this effort around leveraging high-density surface EMG and IMU to detect compulsions. We can then use EDA and PPG signals to examine the preceding obsessional states associated with the uncontrolled urge to compulse.

Finally, a limiting factor in prior efforts is the lack of OCD symptom tracking and real-time modeling—we need to infer symptom state from sensor data as it arrives. FIG. 3D illustrates the approach we propose for backend development to support use of wearables outside of the lab. Commercial wearables natively stream sensor data to mobile applications using standard wireless protocols like Bluetooth Low Energy (BLE). However, custom applications are typically required for continuous streaming and data logging. State-of-the-art machine learning (ML) and artificial intelligence (AI) and the associated data ecosystems are, however, rapidly advancing. Cloud-based platforms, such as Amazon Web Services (AWS) and Google Cloud, can now store data uploaded from mobile devices during ambulatory use, train and update flexible models to perform feature extraction and symptom classification, and download the models onto the app. Vendors can provide suitable backend tools and user interfaces for custom implementations in real-time as part of remote clinician-assisted telehealth sessions.

Today, OCD symptoms are generally treated behaviorally using exposure and response prevention (ExRP), drugs (SRIs), and in severe intractable cases neuroablation and DBS. Improvements are measured using the Y-BOCS to generate a multifactorial psychometric score that spans subjective and objective aspects of distress and impairment. In addition to Y-BOCS improvements, ExRP and DBS treatment of OCD can improve anxiety and depression symptoms, as measured by the Hamilton Anxiety Rating Scale (HARS) and the Hamilton Depression Rating Scale (HDRS) clinical interviews as well as self-administered measures such as DASS-21. In related work focused on food compulsions, as well as clinical epilepsy, symptomatic monitoring, interventions have also relied on self-reported diaries. However, the poor reliability of self-reporting has been well established with under-reporting rates approaching 50%, depending on the diagnosis, often due to lack of awareness. The advent of both wearable symptom detection technologies and implantable responsive neurostimulation devices has led to significant improvements in record accuracy, and shows promise for probabilistic symptom forecasting for seizure disorder. Technologies to measure symptom frequency and severity for OCD promise similar improvements and could open the door to forecasting obsessive and compulsive events, with the ultimate goal of interrupting compulsions to reduce functional impairment.

Physiologically, OCD is linked to dysregulated corticobasal ganglia networks. While the cascade of neural activity changes that occur during the evolution of OCD symptoms remains unclear, the most severe OCD cases feature hours of daily episodes. This implicates overlapping neural circuits such as those that normally mediate habits and automated behaviors. In OCD and related disorders, these circuits may become hyperactive or inaccessible to a stop signal. Pharmacological treatment of OCD using SRIs in combination with other medications can improve treatment resistant cases. However, the most severely affected patients remain disabled despite pharmacological treatments and are suitable for DBS treatment (neuroablation is an alternative that does not involve an implanted device). Effective DBS targets for OCD seek to capture prefrontal, anterior cingulate, and basal ganglia connections within the limbic system and can deliver substantial ˜43% Y-BOCS reduction. DBS of the ventral anterior limb of the internal capsule and adjacent ventral striatum (VC/VS), where more thalamocortical fibers pass, leads to a gradual improvement in OCD symptoms after 3 months, consistent with better manipulations of the cortico-striato-thalamo-cortical loop. New directions in DBS for treatment-refractory OCD (trOCD) are also being pioneered by MPI Halpern and our team using closed-loop stimulation technology derived from seizure control.

Our innovations stem from computational frameworks used to integrate/synchronize multimodal data streams. Smartsleeve brings together BMI and DBS in an adaptive closed-loop recording and stimulation paradigm. Leveraging behavioral measures will enhance rigor and reproducibility associated with neural signal analysis in ambulatory patient settings. Developing modeling paradigms for long time data series, hours, across populations of patients in a disease-relevant manner will also drive forward clinical innovations in patient care by bringing the latest advances in computational neuroscience and neurotechnology. Our project incorporates novel approaches and advances existing approaches to quantifying behavior, cognition, and changes in internal states in naturalistic human behavior. Specific innovations arise from novel methods and approaches used to integrate complex behaviors with neural data by triggering specific epochs for neural recordings and performing these recordings routinely, each day, for tens of days per patient. This approach will advance understanding of how the brain gives rise to human behavior such as compulsions, based on cognitive processes involving habits, urges, and with respect to changes in internal states for patients in OCD. Unlike existing efforts which only measure neural activity when patients choose to click or swipe, triggering recordings to symptoms like before, during and after the compulsion, will give the opportunity for an entirely new view of the transformation across obsession and compulsion. Key questions are whether high-resolution SmartSleeve tracking can help resolve these interactions, leading to novel biomarkers, and whether higher bandwidth neural data from these circuits-phase not just amplitude-resolve these interactions, and functionally in terms of patient-reported EMAs and clinical measures (Y-BOCS, HARS, HDRS, DASS-21). The SmartSleeve will 1—reveal the temporal dynamics of symptom episodes by sampling physiology and behavior on a sub-second timescale; 2—increase the temporal resolution of self-reports based on EMAs. Fine-grain temporal signatures will be available longitudinally, 8 weeks. The combination of physiological and EMA data offers innovative opportunities for a new understanding of OCD behavior and treatment.

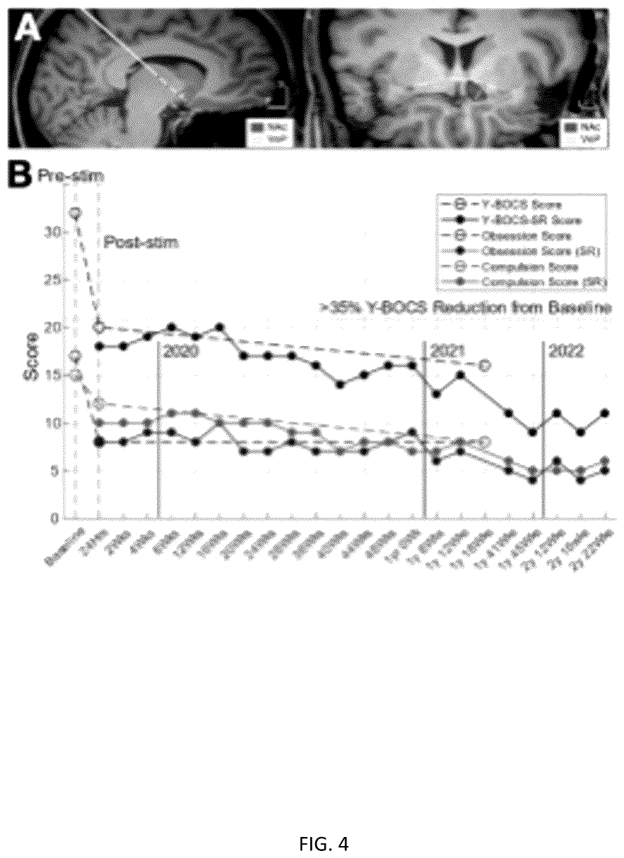

We have robust data from a first-in-human application of closed-loop DBS for trOCD by stimulating sites within the ventral striatum for OCD treatment (FIG. 4A). Findings from this case are profound, as the it was uncovered a reliable biomarker across ambulatory timestamps much like is proposed in the present application, suggesting rigor of this electrographic feature. The responsive nature of the team's stimulation strategy in this case further implicates causality of this biomarker in transitions from obsessions to compulsions, suggesting a common gray matter node within OCD circuitry. Moreover, we algorithmically found a way to ensure this biomarker was detected automatically. This case study demonstrates reduction not only in patient-reported stress scores (SUDS) but also her Y-BOCS, and the improvement was relatively immediate (unlike conventional DBS) and long-lasting (FIG. 4B). Importantly, analyses of invasive iEEG activity that triggered stimulation delivery confirmed that low frequency oscillatory power was the greatest contributor to the signal fluctuations driving signal detections. These data highlight the significance of resolving dynamic network interactions between emotional circuits and cognitive control circuits in delivering DBS. Many neuroscientific studies, including from our own group, also show that the phase of low-frequency iEEG and LFP activity on implanted electrodes is especially important.

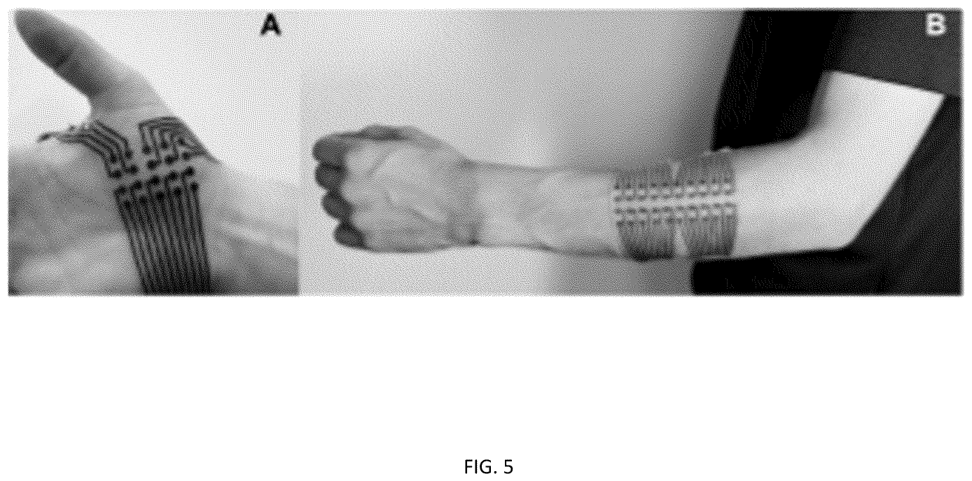

There is developed a novel technology for high-resolution and sensitivity dry surface EMG (sEMG) that we have integrated into prototype SmartSleeve devices. These sEMG electrodes are composed of Ti₃C₂Tₓ MXene, a carbon-based 2D material with enhanced conductivity, electrochemical impedance, and mechanical compliance, compared to metals and Ag/AgCl, making it ideally suited to sensing body bioelectrical activity. Compared to conventional electrode materials, Ti₃C₂Tₓ MXene (or MXene for short) is cheaper to source and process and has 4-10× lower impedance, which translates into higher sensitivity and better signal-to-noise characteristics without the need for any gel or harsh skin treatment. There was developed a high throughput, low-cost and scalable process to fabricate MXene-based dry sEMG sensors. By combining laser machining of absorbent textile substrates with printing of MXene water-based inks and silicone encapsulation, we can fabricate electrodes with fully customizable size, geometry, and density (FIG. 5). Owing to the low impedance and high conformability with the skin, these sEMG electrodes do not require any conductive gel to operate, are not subjected to movement artifacts, and can detect muscle activity with high sensitivity and resolution comparable to wireless commercial sEMG sensors (i.e., Delsys Trigno). Finally, these arrays can be used for continuous monitoring, as the impedance with the skin did not vary during continuous wearing on the forearm for >2 days.

We have developed electronics for a prototype SmartSleeve with most of the proposed hardware functionality necessary for this proposal (8-channel EMG, 6-axis IMU, and PPG) (FIG. 2). The 1.0×0.5 inch device can wirelessly transmit the data, sampled at 1 kS/s/ch, to a Bluetooth receiver >2 m away and operate continuously for >4 h using a 4.6 g rechargeable battery. The prototype Sleeve arises from a 10+ year collaboration between Co-Is Liu and Richardson to develop battery-powered wireless systems with discrete off-the-shelf systems and custom fully integrated circuits, such as wireless sensor nodes to measure behavioral variables, grip force and joint angles, for closed-loop BMIs. The prototype SmartSleeve electronics are similar in design to their prior battery-powered, printed circuit board (PCB)-based wireless sensor nodes with an analog front end (AFE) to interface with sensors, microcontroller (MCU) for data digitization and processing, and backend 2.4 GHz RF transceiver for wireless data transmission.

We have obtained a non-significant risk designation for SmartSleeve studies under IRB approval from the Office of Clinical Research at UPenn. We have enrolled a patient (Y-BOCS=29) and performed our first study with the SmartSleeve prototype. We enrolled the patient in the symptom journal. We issued 45 surveys 9 am-7 pm over 14 days with responses within 1 minute of the survey trigger. In response to: “In the past hour, how many minutes have you spent doing any compulsion/ritual?” the patient reported 0-20 min (median: 3.5 min). Responses to “Right now, what are your urges to engage in any compulsion/ritual?” scored 0-80 (mean: 32). Responses to “What is your distress right now?” scored 0-20 (mean: 10). Responses to “Right now, how much effort are you making to resist any compulsion/rituals?” scored 0-80 (mean: 21). For the in-clinic symptom provocation experiment, Dr Brown provoked symptoms as the patient wore the prototype SmartSleeve as well as clinical-grade BIOPAC sensors (respiration, heart beat, skin resistance). Repeated trials involving provocation of distress and urges to compulse, performance of compulsive behavior to reduce distress and urges, and performance of non-compulsive ADL were completed (FIG. 2).

SmartSleeve data was streamed to a laptop throughout the session. Here, we analyze the EMG data to detect compulsions from ADL using standard approaches. We band-pass filtered, 50-450 Hz, and rectified EMG activity. We then computed root-mean-square power of EMG activity on each channel over a feature sample window of short duration. We then concatenated EMG activity from 50% overlapping feature sample windows in a longer feature integration window. We reduced dimensionality of features in the feature integration window using PCA and selected the top set of modes as features for a logistic regression. Activity from each feature integration window was classified as compulsion or ADL. Feature sample window duration, feature integration window and PCA dimension were metaparameters and selected by analyzing a validation data set. FIG. 2 parameters are feature sample window=6 s. feature integration window=40 s. PCA dimension=3. We estimate accuracy of the classifier given by the average of sensitivity and specificity using cross-validation with a test set. Accuracy for effective compulsions was 80% and for ineffective compulsion was 52%. FIG. 2 left presents data projected using t-SNE, a nonlinear data visualization method. We show a point for each feature integration window on trials with effective compulsions (red) or ADL (green). FIG. 2 right presents the same for trials with ineffective compulsions (orange) or ADL (green). This suggests that our experimental design can reveal the potential power of the SmartSleeve technology to detect effective compulsions with accuracy exceeding 75%.

### Design

The SmartSleeve can be a battery-powered, wireless custom arm sleeve with multimodal sensors to record markers of obsessions and compulsive behaviors. To quantify anxiety, the combination of HRV (as recorded with PPG) and electrodermal activity (EDA), along with accelerometry (as recorded with an IMU) to clean EDA artifacts during movement, provides the best performance. To quantify compulsive behaviors, which most often involve manual actions, we selected multichannel forearm EMG and again accelerometers and gyroscopes provided by an IMU.

In some cases, 8 bipolar EMG channels can be sufficient to capture hand movements in sufficient detail. These signals can be acquired using an Intan RHD amplifier chip at 1000 samples/s/ch. The RHD also has an alternating current (AC) impedance measurement feature. We will use this feature intermittently to both measure electrode-skin impedance to evaluate proper SmartSleeve use, as well as to acquire AC-based EDA measurements from the same electrodes used for EMG. The EMG and EDA outputs from the RHD chip, along with the digital outputs from a 6-axis IMU and a PPG module, can be routed to the MCU that contains both a 128 MHz Arm Cortex processor and a RF transceiver module for wireless data transmission to the smartphone app. The PCB can also drive a vibration motor to prompt EMA feedback through two integrated pushbutton switches. 3.3 V regulators (Vreg) can perform power management. Total power consumption of the device can be less than 90 mW, allowing a rechargeable 500 mAh Li-ion battery weighing 8 g to power the device continuously for over 18 h. The estimated SmartSleeve data rate is less than 300 kb/s, well within the maximum BLE 5.0 rate of 2 Mb/s.

The PCB and battery can be secured within pouches in an elastic textile sleeve, with an external connector for recharging. The EMG electrodes can be positioned within the sleeve to record from large and small flexor and extensor forearm muscles. The EMG electrodes can be fabricated based on MXene-textile technology. Arrays from our preliminary work had 8 contacts. Here, we can fabricate arrays of 16 contacts (diameter: 8 mm) to maximize spatial sampling of the forearm and robustness. The arrays can be embedded in a perforated silicone encapsulation for comfort and breathability, while ensuring conformal adhesion with the skin and easy integration in the elastic textile sleeve. Based on our preliminary data, with proper skin prep, electrode-skin impedance at 10 Hz and 1 kHz can be on the order of 50 and 10 kΩ, respectively and can remain stable for the targeted 3 hours of continuous use.

### User Interface on the Device

An EMA labeling interface can be incorporated into the sleeve to label symptom epochs and rest epochs during spontaneous recording experiments. A goal is to parsimoniously label compulsion/ritual behaviors synchronized with SmartSleeve signals. Two low-profile pushbutton switches can be incorporated into the sleeve, with thumbs up/thumbs down icons. At any time in the session, patients can select these responses to indicate whether they are engaging in compulsive behaviors. Patients can also be prompted using a vibration motor controlled by the app. Patients can be asked to provide a compulsion yes/no EMA that can be used to validate accuracy of the SmartSleeve symptom detector. Privacy mode can be activated by simultaneously pressing both buttons. Alternatively, EMA can be moved to the smartphone app.

In some cases, the sleeve can include PPG, EDA, IMU, and EMG sensors with continuous wireless data collection for at least 18 h on a single charge, a wireless bit error rate of <0.001%, and a EMG signal-to-noise ratio (SNR) of at least 15 dB with expected impedance at 10 Hz and 1 kHz will be ˜50 and <10 kΩ.

An app running on a dedicated mobile device can be provided to patients that will acquire streamed data from the SmartSleeve wireless transmitter on the arm. Data can be hardware encrypted to ensure security during transfer between the Sleeve and the App. The firmware can continuously acquire the Sleeve data stream sent via Bluetooth protocol as well as acquire the less-frequent EMA labels on the Sleeve. Users can input EMA labels whenever they choose to provide a rating. Users can be requested to respond to queries to provide EMA labels by the device, for example by buzzing. In the app, EMAs can be integrated with the electronic symptom journal the patients already keep. EMG activity can be preprocessed. IMU data can be preprocessed to sample at 10 Hz. The EDA signal can be preprocessed by low-pass filtering at 2 Hz and sampling at 4 Hz. Sweat secretion process can be modelled using 3D state-space representation and consider the underlying sparse arousal events as stimuli. The phasic component can be separated from the tonic component and deconvolve the underlying arousal events via an Expectation-Maximization based approach. PPG data can be preprocessed to detect each heartbeat and measure heart-rate variability as the inter-beat time approx. once per second. The short-time, ˜0.1-1 s, feature samples can be pooled into longer 10-100 s duration feature integration periods. The feature integration windows can be chosen to capture periods of high compulsivity behaviors that extend longer than comparison movements and activities of daily living. Neighboring windows can overlap in time, approximately 10% of the window size.

A classifier can be trained to generate output triggers from each feature integration window time. Training labels can either come from patient-reported SUDS and urges ratings during the symptom provocation experiments or patient-generated EMA labels during spontaneous recording experiments. The SmartSleeve classifier can calculate the probability of having the OCD state higher than the patient's baseline. When running in real-time, the classifier can also trigger recordings of neural activity. Sensor data/EMAs can be synced to an AWS repository for model training and storage. Models can be personalized to each patient and downloaded to the app using standard cloud computing workflows.

### SmartSleeve+Platform

The SmartSleeve+ extends the SmartSleeve platform to record neural data from implanted DBS leads. Development centers on firmware upgrades to the DBS IPG to access iEEG activity, the wearable coil dongle for data streaming and integration with SmartSleeve App.

The existing DBS System delivers high-frequency stimulation compatible with standard 1×4 extensions and iEEG recordings from 1-35 Hz. FIG. 3A presents the system which features a Patient controller containing a coil that provides an inductive link to activate the IPG for recharge mode. The RF link is used to handshake and check charging performance. The Physician controller can connect to the IPG via the same coil-based RF link for reprogramming the stimulator.

In the new system (FIG. 3B), a Coil Dongle can be implemented to use an inductive link to activate the IPG and RF link for real-time streaming mode. The Coil Dongle RF link can also support reprogramming the stimulator, for example to turn on or off stimulation.

This functionality will enable future studies to perform adaptive DBS. Specific firmware can be implemented for event-driven signal streaming on the IPG triggered by the SmartSleeve+ coil dongle. A small (e.g., 50 mm diameter) coil sits in a customized project-provided undergarment worn by the user that will communicate with the SmartSleeve app to receive triggers and stream iEEG activity (2 channels, 128 Hz sampling rate, 16 bits/sample) from implanted leads connected to the DBS IPG. The Coil Dongle can contain a battery, an inductive coil, microcontroller, solid state storage for data transfer (FRAM) and a bluetooth interface. The bluetooth interface can receive software triggers and send commands to an inductive coil that will trigger neural recordings from implanted DBS leads. The trigger activates the inductive coil. The inductive coil injects current to wake up the IPG with a START streaming mode command. Within 1 s, raw iEEG activity on the leads can then continuously stream via an RF link to the Coil Dongle until receiving a STOP command. The STOP command can occur at any time after the START command, subject to IPG battery life. After the STOP command, streaming mode will halt. The IPG can then transfer the entire contents of the FRAM buffer which contains the raw iEEG activity immediately before the START command was issued. Finally, the IPG can send the battery state. This can record raw iEEG activity for a fixed period, e.g., up to 4 minutes, before the trigger and a variable period, constrained by IPG battery life, after the trigger. With reasonable Coil-IPG placement, data transfer rate from the IPG to the Coil Dongle is 500 kbit per second. With negotiation of transfer and other processes we can flush 1 Mbit memory—raw signals and biomarkers—in ˜1 minute. This time occurs at the end of the streaming mode. After the memory is flushed, data acquisition to the raw signal buffer resumes.

Data can be streamed to the SmartSleeve+ app using the RN4870 BLE module. The SmartSleeve App can trigger neural recordings based on specified events e.g., triggered by the Sleeve data, triggered by the user using Sleeve EMA buttons, and triggered by symptom journal surveys. The app can synchronize neural data with SmartSleeve data.

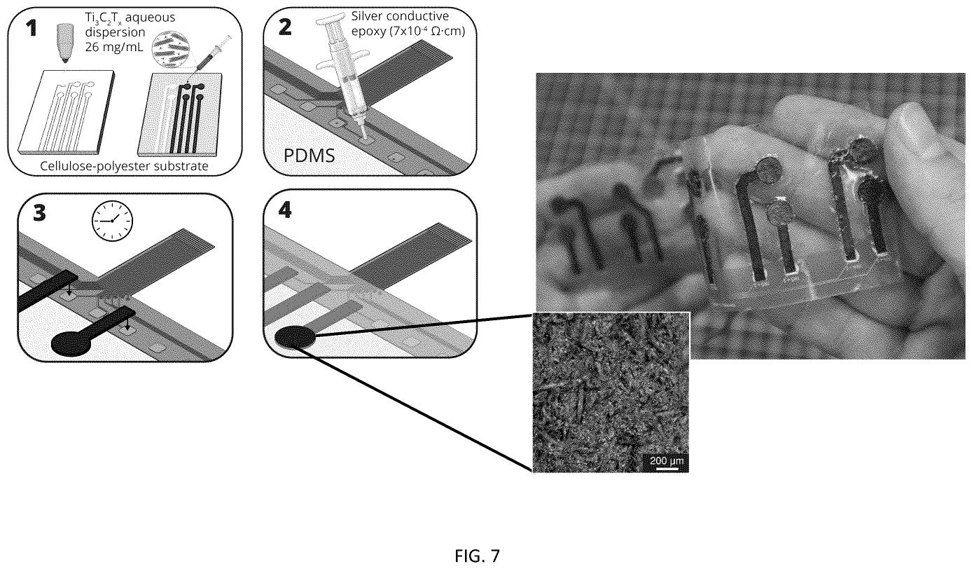

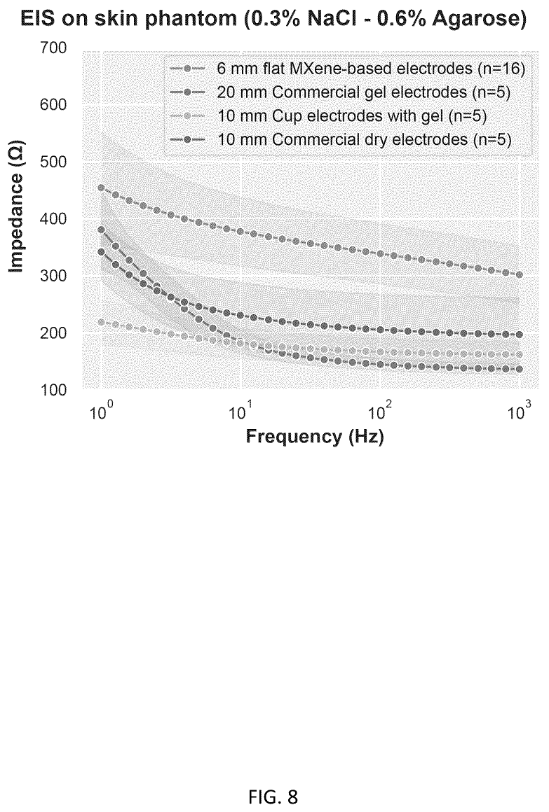

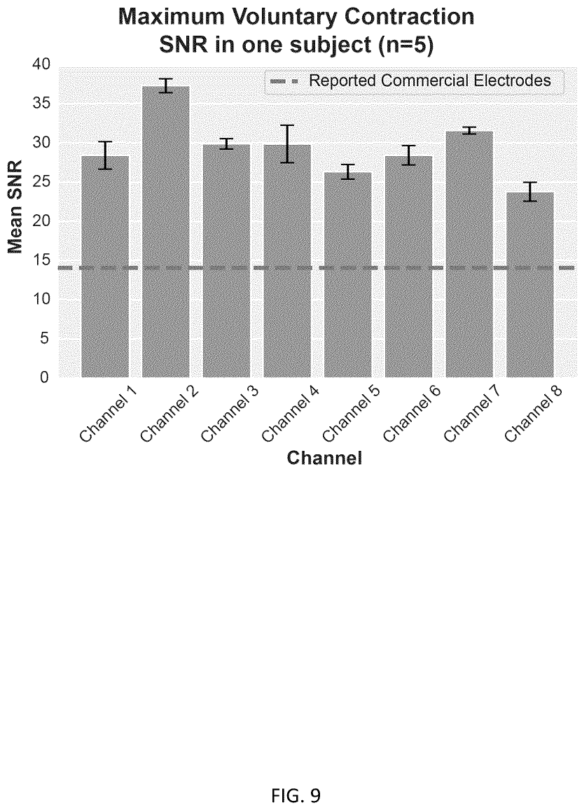

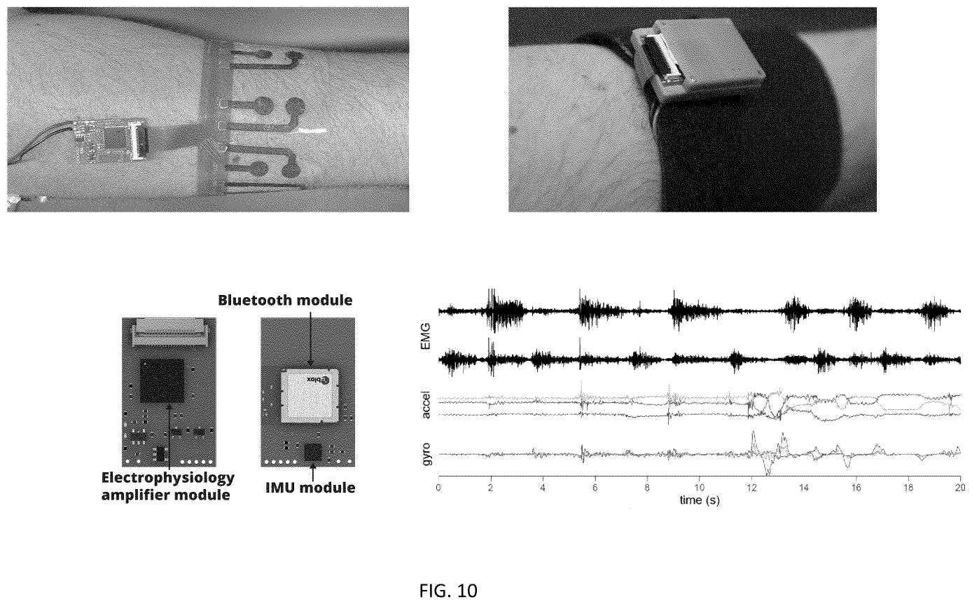

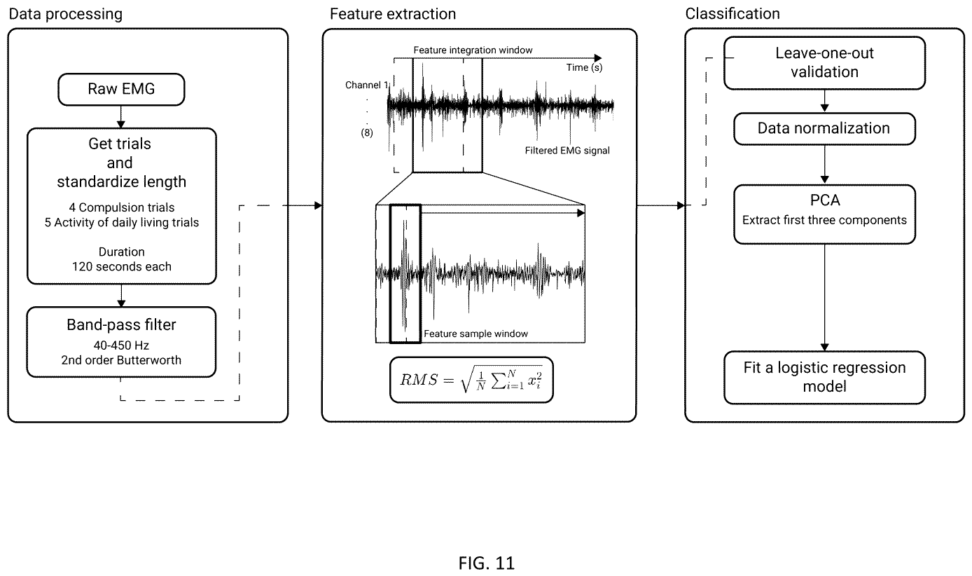

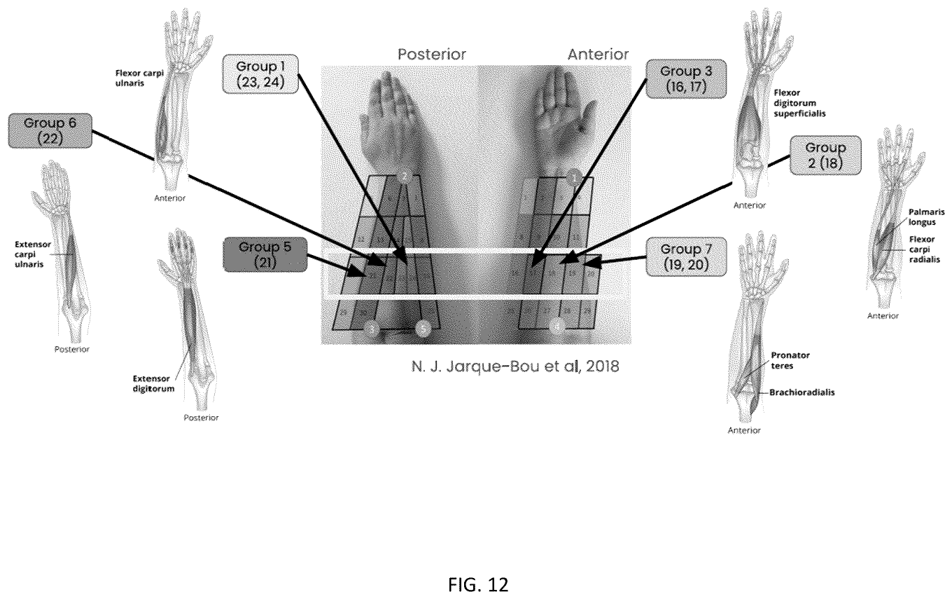

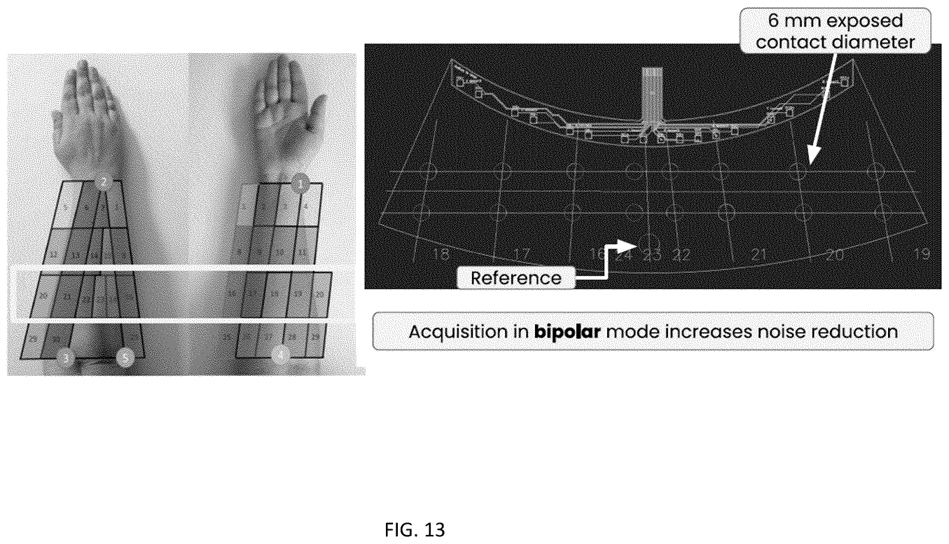

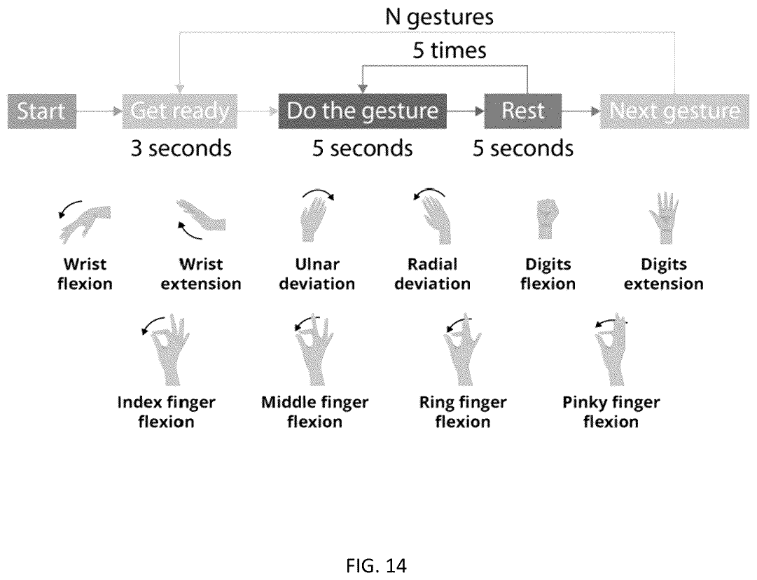

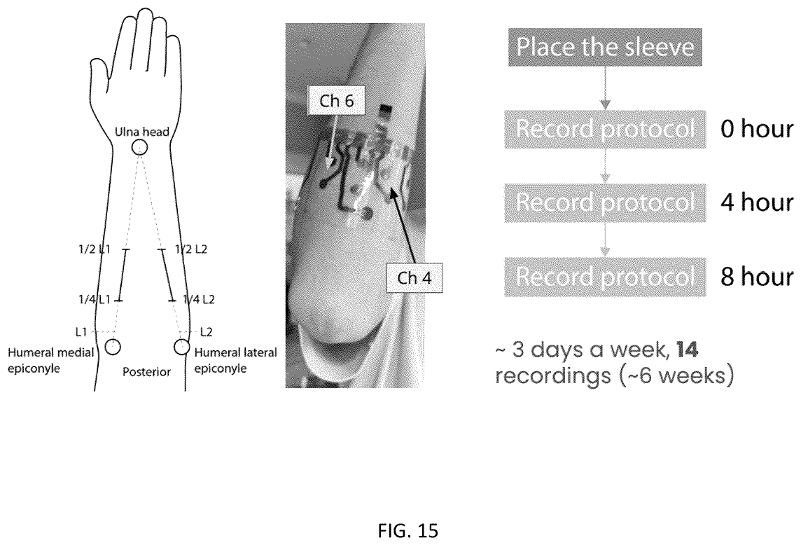

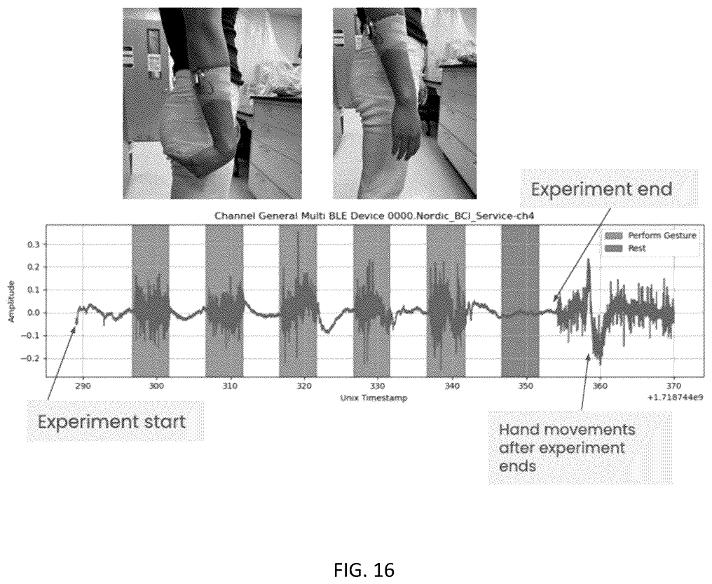

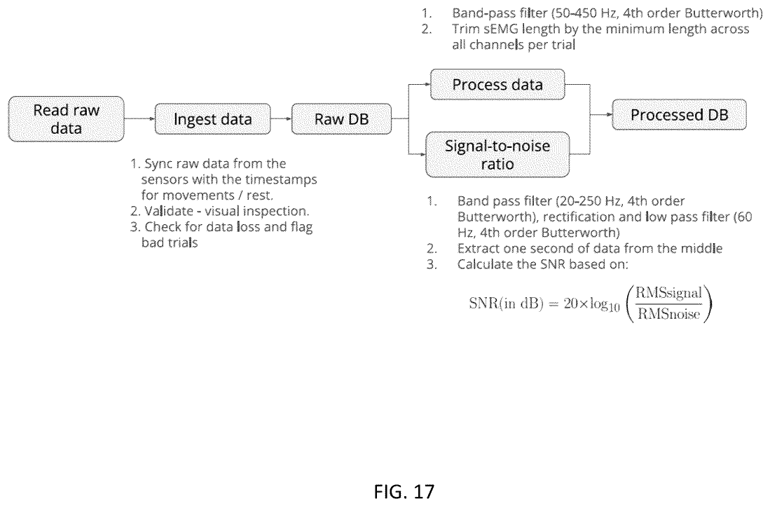

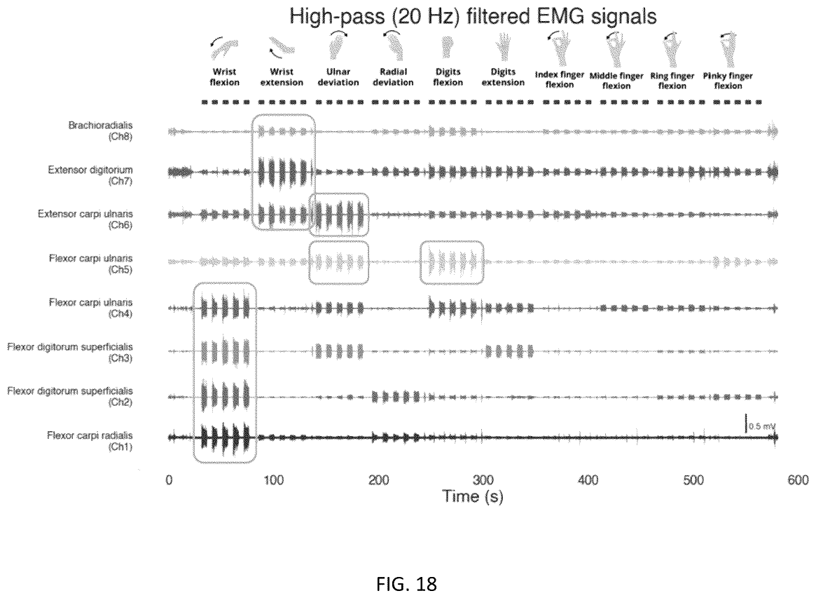

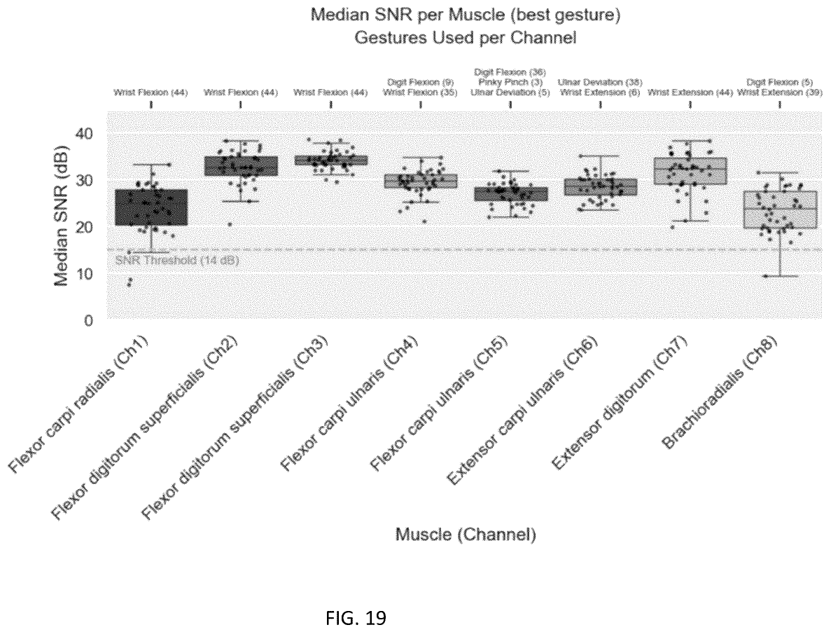

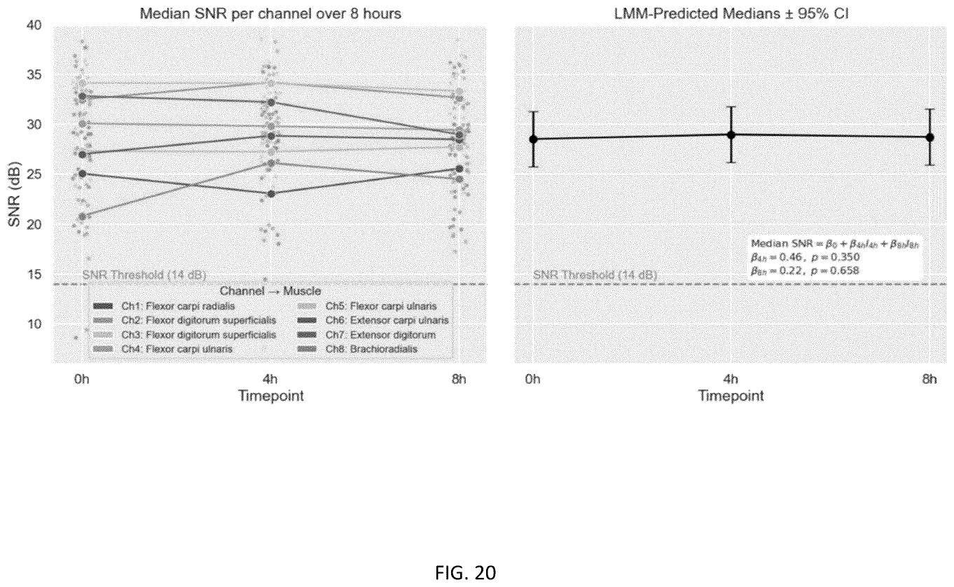

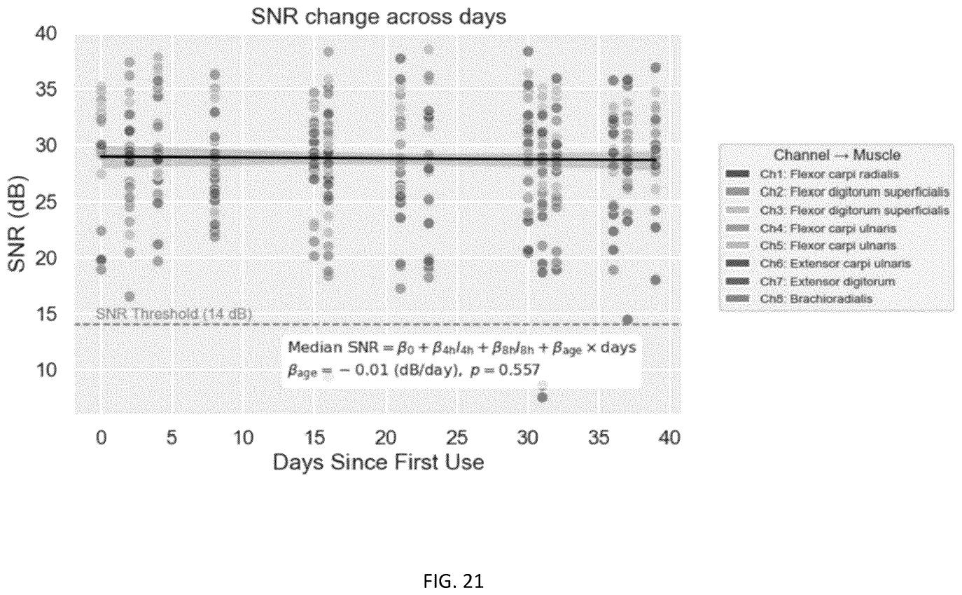

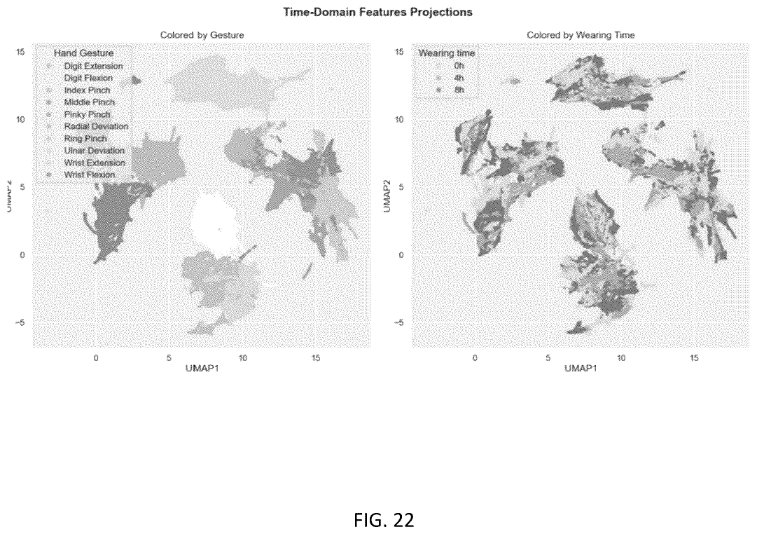

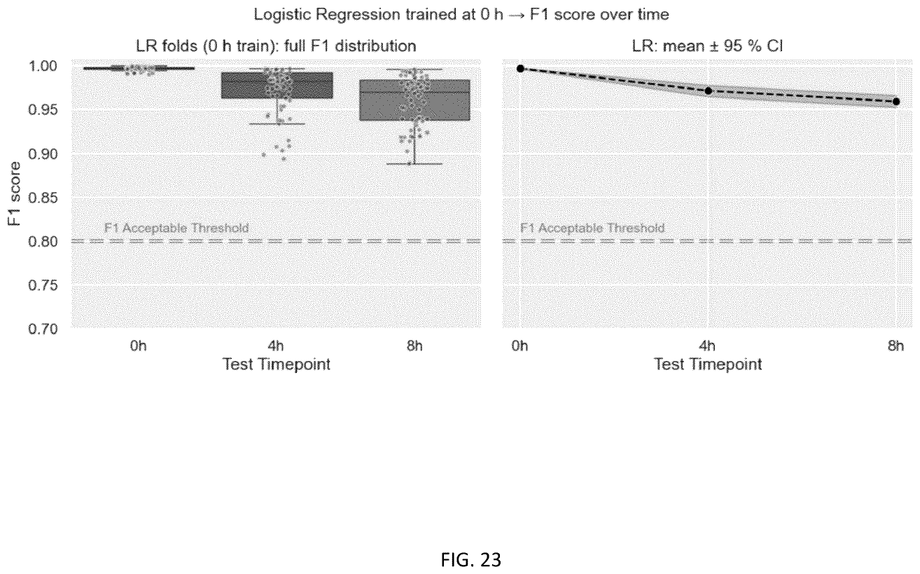

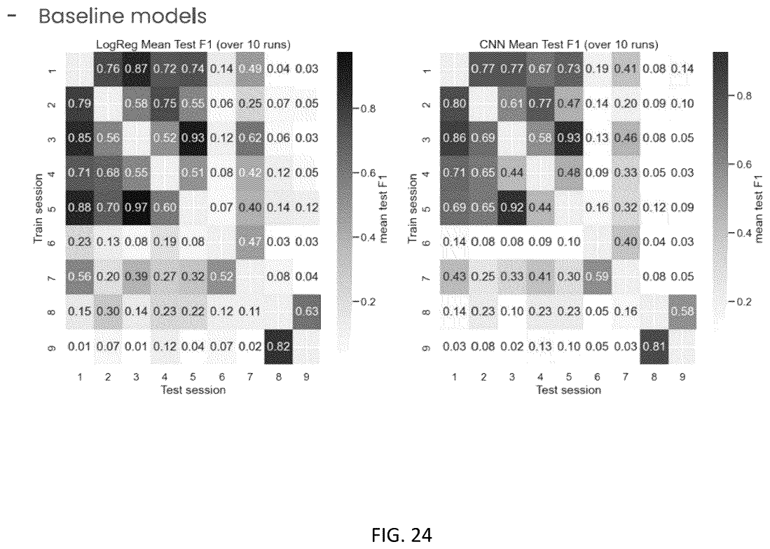

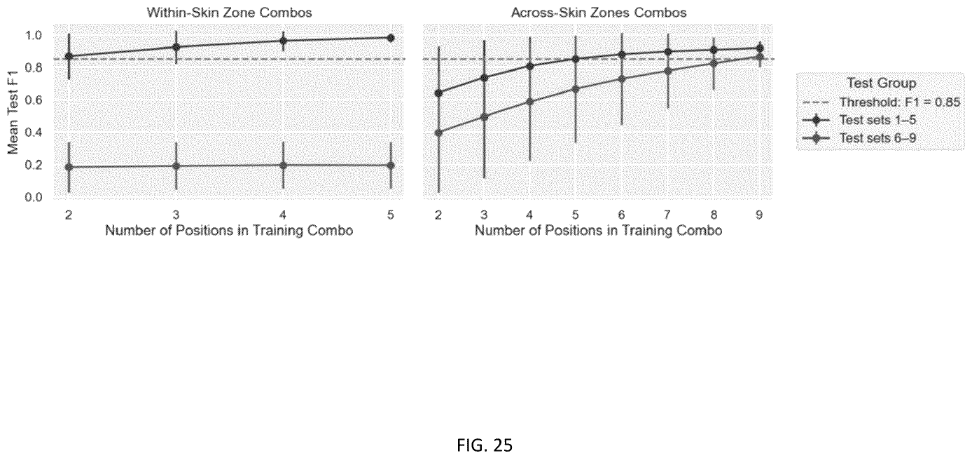

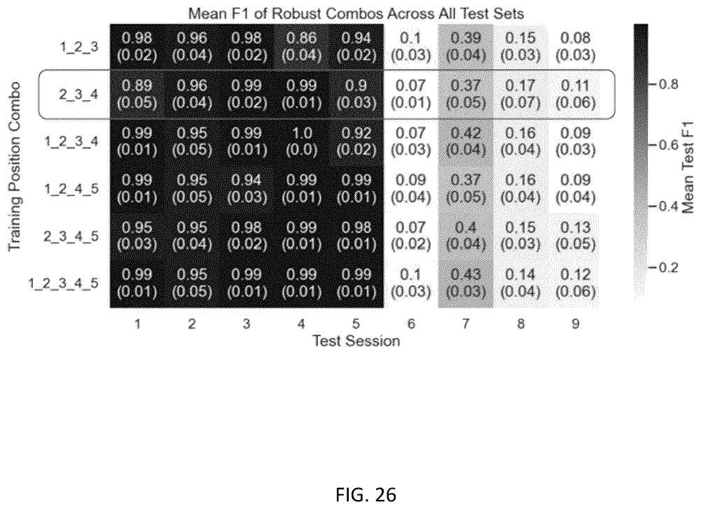

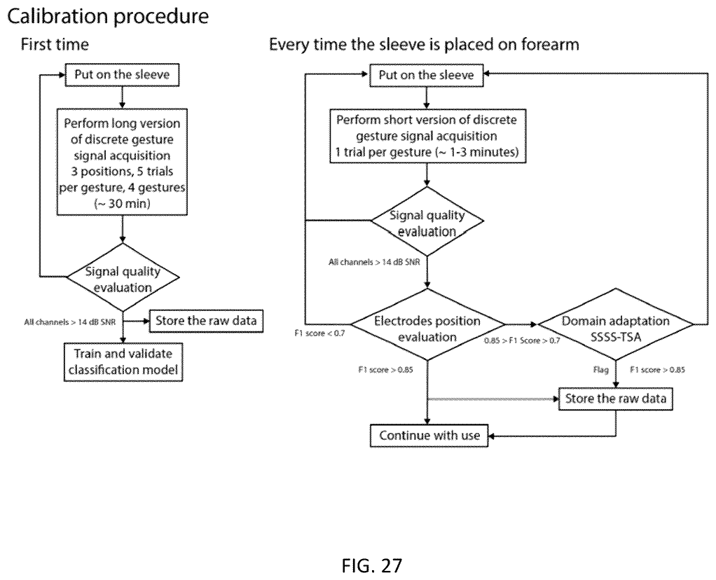

FIG. 7 shows examples of MXene electrodes according to the present disclosure. FIG. 8 shows a graph of impedance vs. frequency for different electrode examples. FIG. 9 shows a graph of mean signal-to-noise ratio (SNR) for corresponding channels of MXene-based electrodes according to the present disclosure. FIG. 10 shows an example wireless acquisition device that collects and transmits sEMG and inertial data according to the present disclosure. FIG. 11 shows a data processing system using a nested sliding window to classify effective compulsions according to the present disclosure. FIG. 12 shows electrode placement based on forearm zones. FIG. 13 shows zone mapping and corresponding electrode array design. FIG. 14 shows a discrete hand gesture acquisition protocol. FIG. 15 shows a recording process for electrodes. FIG. 16 shows SNR measurement for electrode system. FIG. 17 shows a data analysis pipeline. FIG. 18 shows high-pass filtered EMG signals. FIG. 19 shows median SNR per muscle gestures used per channel. FIG. 20 shows median SNR per channel, and corresponding predicted medians. FIG. 21 shows SNR change across a number of days. FIG. 22 shows time-domain features projections. FIG. 23 shows classification performance over time. FIG. 24 shows baseline models for electrode shift compensation. The process included processing raw sEMG data (50-450 Hz) band-pass Butterworth filter (4^{th} order), storing the processed data locally to be loaded by a trainer. For each training session: data splitting: take one position (randomly selecting one trial for validation) and one position for testing-pairwise; data segmentation: segment each trial in windows of 250 ms with 225 ms overlap; normalize the data by channel using a training scaler; and wrap in pytorch data loaders. Trained two models: logistic regression (repeat each training 10 times) and 1D CNN (set to 5 epochs and run 5 times). FIG. 25 shows graphs of electrode shift experiments: Multi-position electrode placement within the same skin zone. 4 trials for each position were taken for training (1 for validation) and 1 trial from other positions for test (pairwise). The data was randomized and repeated 10 times. FIG. 26 shows a graph corresponding to electrode shift experiments. The results suggest that the model can accurately predict if the sleeve is placed within the skin zone range. FIG. 27 shows a calibration process.

### Exemplary Embodiments

The following embodiments are exemplary only and do not serve to limit the scope of the present disclosure of the appended claims. It should be understood that any part of any one or more Embodiments can be combined with any part of any other one or more Embodiments.

### Embodiment 1

A method, comprising: measuring, via a wearable device of a diagnostic system, one or more electrical signals corresponding to muscle movement of a user, wherein the wearable device comprises one or more MXene-comprising sensors; inputting information indicative of the one or more electrical signals into a trained model; determining, by the trained model and based on the inputted information, whether the user is experiencing symptoms of a neuropsychiatric disorder; and sending information indicative of the determining to a component of the diagnostic system.

### Embodiment 2

The method of Embodiment 1, further comprising: causing a neural recording to be performed based on determining the user is experiencing symptoms of a neuropsychiatric disorder.

### Embodiment 3

The method of Embodiment 2, wherein the neural recording is caused by an implantable pulse generator (IPG).

### Embodiment 4

The method of any of Embodiments 1 to 3, further comprising: causing a neurostimulation to be performed based on determining the user is experiencing symptoms of a neuropsychiatric disorder.

### Embodiment 5

The method of Embodiment 4, wherein the neurostimulation is caused by an implantable pulse generator (IPG).

### Embodiment 6

The method of any of Embodiments 1 to 5, wherein the determining further comprises: identifying whether the inputted information corresponds to activities of daily living (ADL) or symptoms of a neuropsychiatric disorder.

### Embodiment 7

The method of any of Embodiments 1 to 6, wherein the neuropsychiatric disorder comprises any one or more of obsessive compulsive disorder (OCD), Tourette's syndrome, or Parkinson's disease.

### Embodiment 8

The method of any of Embodiments 1 to 7, wherein the wearable device comprises a sleeve configured to be worn on an arm of the user.

### Embodiment 9

The method of any of Embodiments 1 to 8, wherein the wearable device further comprises one or more inertial measurement unit (IMU) sensors, and wherein the method further comprises measuring acceleration information of the wearable device, wherein the determining is further based on the acceleration information.

### Embodiment 10

The method of any of Embodiments 1 to 9, wherein the one or more electrical signals comprise electromyography signals.

### Embodiment 11

A system, comprising: a wearable device comprising one or more MXene-comprising sensors, wherein the wearable device is configured to measure one or more electrical signals corresponding to muscle movement of a user; a trained model configured to: receive information indicative of the one or more electrical signals; and determine, based on the inputted information, whether the user is experiencing symptoms of a neuropsychiatric disorder; and a component configured to receive information indicative of the determining.

### Embodiment 12

The system of Embodiment 11, further comprising a second component configured to: cause a neural recording to be performed based on determining the user is experiencing symptoms of a neuropsychiatric disorder.

### Embodiment 13

The system of Embodiment 12, wherein the second component comprises an implantable pulse generator (IPG).

### Embodiment 14

The system of any of Embodiments 11 to 13, further comprising a second component configured to: cause a neurostimulation to be performed based on determining the user is experiencing symptoms of a neuropsychiatric disorder.

### Embodiment 15

The system of Embodiment 14, wherein the second component comprises an implantable pulse generator (IPG).

### Embodiment 16

The system of any of Embodiments 11 to 15, wherein the determining further comprises: identifying whether the inputted information corresponds to activities of daily living (ADL) or symptoms of a neuropsychiatric disorder.

Embodiment 17

The system of any of Embodiments 11 to 16, wherein the neuropsychiatric disorder comprises any one or more of obsessive compulsive disorder (OCD), Tourette's syndrome, or Parkinson's disease.

### Embodiment 18

The system of any of Embodiments 11 to 17, wherein the wearable device comprises a sleeve configured to be worn on an arm of the user.

### Embodiment 19

The system of any of Embodiments 11 to 18, wherein the wearable device further comprises one or more inertial measurement unit (IMU) sensors, and wherein the wearable device is further configured to measure acceleration information of the wearable device, wherein the determining is further based on the acceleration information.

### Embodiment 20

The system of any of Embodiments 11 to 19, wherein the one or more electrical signals comprise electromyography signals.

What is claimed:

1. A method, comprising:
measuring, via a wearable device of a diagnostic system, one or more electrical signals corresponding to muscle movement of a user, wherein the wearable device comprises one or more MXene-comprising sensors;
inputting information indicative of the one or more electrical signals into a trained model;
determining, by the trained model and based on the inputted information, whether the user is experiencing symptoms of a neuropsychiatric disorder; and
sending information indicative of the determining to a component of the diagnostic system.

2. The method of claim 1, further comprising:
causing a neural recording to be performed based on determining the user is experiencing symptoms of a neuropsychiatric disorder.

3. The method of clam 2, wherein the neural recording is caused by an implantable pulse generator (IPG).

4. The method of claim 1, further comprising:
causing a neurostimulation to be performed based on determining the user is experiencing symptoms of a neuropsychiatric disorder.

5. The method of claim 4, wherein the neurostimulation is caused by an implantable pulse generator (IPG).

6. The method of claim 1, wherein the determining further comprises:
identifying whether the inputted information corresponds to activities of daily living (ADL) or symptoms of a neuropsychiatric disorder.

7. The method of claim 1, wherein the neuropsychiatric disorder comprises any one or more of obsessive compulsive disorder (OCD), Tourette's syndrome, or Parkinson's disease.

8. The method of claim 1, wherein the wearable device comprises a sleeve configured to be worn on an arm of the user.

9. The method of claim 1, wherein the wearable device further comprises one or more inertial measurement unit (IMU) sensors, and wherein the method further comprises measuring acceleration information of the wearable device, wherein the determining is further based on the acceleration information.

10. The method of claim 1, wherein the one or more electrical signals comprise electromyography signals.

11. A system, comprising:
a wearable device comprising one or more MXene-comprising sensors, wherein the wearable device is configured to measure one or more electrical signals corresponding to muscle movement of a user;
a trained model configured to:
receive information indicative of the one or more electrical signals; and
determine, based on the inputted information, whether the user is experiencing symptoms of a neuropsychiatric disorder; and
a component configured to receive information indicative of the determining.

12. The system of claim 11, further comprising a second component configured to:
cause a neural recording to be performed based on determining the user is experiencing symptoms of a neuropsychiatric disorder.

13. The system of clam 12, wherein the second component comprises an implantable pulse generator (IPG).

14. The system of claim 11, further comprising a second component configured to:
cause a neurostimulation to be performed based on determining the user is experiencing symptoms of a neuropsychiatric disorder.

15. The system of claim 14, wherein the second component comprises an implantable pulse generator (IPG).

16. The system of claim 11, wherein the determining further comprises:
identifying whether the inputted information corresponds to activities of daily living (ADL) or symptoms of a neuropsychiatric disorder.

17. The system of claim 11, wherein the neuropsychiatric disorder comprises any one or more of obsessive compulsive disorder (OCD), Tourette's syndrome, or Parkinson's disease.

18. The system of claim 11, wherein the wearable device comprises a sleeve configured to be worn on an arm of the user.

19. The system of claim 11, wherein the wearable device further comprises one or more inertial measurement unit (IMU) sensors, and wherein the wearable device is further configured to measure acceleration information of the wearable device, wherein the determining is further based on the acceleration information.

20. The system of claim 11, wherein the one or more electrical signals comprise electromyography signals.
